(19) United States
(12) Patent Application Publication
(10) Pub. No.: US 2012/0253401 A1
(43) Pub. Date: Oct. 4, 2012

(54) VERTEBRAL COLUMN IMPLANT FOR STABILIZATION AND STIFFENING OF VERTEBRAL BODIES OF A VERTEBRAL COLUMN

(75) Inventors: Jonathan CLARK, Kloten (CH); Reto Braunschweiler, Neftenbach (CH); Thomas Zehnder, Bach (CH)

(73) Assignee: Spinelab AG, Winterthur (CH)

(21) Appl. No.: 13/431,103

(22) Filed: Mar. 27, 2012

(30) Foreign Application Priority Data
Mar. 31, 2011 (EP) 11160709.9

(51) Int. Cl. A61B 17/70

(52) U.S. Cl. 606/264

(57) ABSTRACT

A vertebral column implant for stabilizing and stiffening of vertebral bodies of a vertebral column comprises at least one first bone screw, screwable into a vertebral body, and at least one second bone screw. A first rigid connecting element, is insertable into first receiving means of the first bone screw. A second elastic connecting element, is insertable into second receiving means of the second bone screw. The connecting means comprise two arms, which form a U-shaped recess. Insertable into a first region of the U-shaped recess is an end region of a first connecting element. Insertable into a second region of the U-shaped recess is an end region of a second connecting element. Inserted between the two arms is a pressing element. Placed on the arms and are clamping means, with which the pressing element is pressed against two end regions of the first and second connecting elements.

This invention relates to a vertebral column implant for stabilization and stiffening of vertebral bodies of a vertebral column, comprising at least one first bone screw having a screw-in part that is screwable into a vertebral body, and first receiving means; at least one second bone screw having a screw-in part that is screwable into a vertebral body, and second receiving means; at least one first connecting element, which is rigid and is insertable into the first receiving means of the first bone screw and is able to be fixed therein; at least one second connecting element, which is elastic and is insertable into the second receiving means of the second bone screw and is able to be fixed therein; and connecting means, with which in each case a first connecting element is connectible to a second connecting element.

Such vertebral column implants for stabilization and stiffening of vertebral bodies of a vertebral column are known in diverse designs. Achieved with the stiffening part of this vertebral column implant is that an osseous growing together between the vertebral bodies stiffened in this way takes place, which leads to a desired stabilization of the vertebral column, while, with the elastic region of the vertebral column implant, the vertebral bodies are stabilized in a supporting way; a certain movability between the individual vertebral bodies is desired and is permitted.

Such a vertebral column implant for stabilization and stiffening of vertebral bodies of a vertebral column is known for example from EP-A-1 961 392.

With these known vertebral column implants, the placement of the bone screws in the individual vertebral bodies, especially in the region of the transition to the rigid connecting element, must take place very precisely. Certain instances of imprecision can be corrected in that the rigid connecting element is suitably bent, which must be accomplished during the insertion of the vertebral column implant in a vertebral column, and which makes the procedure more difficult. Moreover the connecting of the rigid connecting element to the elastic connecting element is time-consuming and involved since each individual connecting element is inserted separately into the respective connecting means and is fixed via an own clamping means, which requires a relatively complicated construction for the corresponding connecting means and which also further complicates the procedure.

The object of the present invention thus consists in creating a vertebral column implant for stabilization and stiffening of vertebral bodies of a vertebral column which is of simple construction, whereby in particular the insertion and connection of the rigid and elastic connecting elements is simplified and with which an optimal connection can be achieved of the rigid connecting elements and the elastic connecting elements.

This object is achieved according to the invention in that the connecting means comprise two arms, which form a U-shaped recess and which are provided on a further bone screw, an end region of a first connecting element is insertable in a first region of the U-shaped recess, and an end region of a second connecting element is insertable in a second region of the U-shaped recess, a pressing element is insertable between the two arms, and clamping means are placeable on the arms, with which clamping means the pressing element is able to be pressed against said two end regions of the first and second connecting element.

Achieved with this design according to the invention is that with a single clamping operation both the rigid connecting element and the elastic connecting element can be clamped and fixed in the U-shaped recess of the further bone screw, whereby the handling is greatly simplified.

Preferably the pressing element is provided with guides which co-operate with guide surfaces disposed on the flanks of the arms, whereby the pressing element can be placed on the arms in a guided way, which simplifies this step.

Preferably the clamping means comprise a clamping screw, with which the pressing element is able to be pressed with a first pressing surface against the end region of the first connecting element and with a second pressing surface against the end region of the second connecting element, which results in a simple construction and a simple clamping procedure.

Preferably the clamping screw is inserted into a cap, which is placeable on the arms and is lockable therewith, whereby an easy insertion of the clamping means in the bone screw is achieved.

This can also be achieved in that the clamping screw is screwable into a threading provided on the inside of the arms.

The clamping means are advantageously formed by a cap which is placeable on the arms and is lockable therewith, and inserted into the cap is a clamping screw, with which the pressing element is able to be pressed with a first pressing surface against the end region of the first connecting element and with a second pressing surface against the end region of the second connecting element, which results in a simple construction for this clamping means and thereby in an easy handling.

Another advantageous embodiment of the invention consists in that the end region of the first connecting element is designed spherical and the bottom surface, formed between the first arm and the second arm, of the first region of the U-shaped recess has a concave spherical shape which corresponds with the spherical end region of the first connecting element. Achieved thereby is that the first connecting element, which is rigid, can be aligned on all sides within a certain angular range with respect to the clamping means, and thus to the second elastic connecting element, and can be adapted optimally to the bone screws in the vertebral bodies, whereby it is achieved that an imprecise placement of the bone screws in the vertebral bodies can be compensated for through the possibility of a polyaxial alignment of the first connecting element instead of through complicated and difficult bending of the rigid connecting element during the procedure.

To obtain an optimal clamping effect and consequently optimal fixing of the connecting elements, the respective pressing surfaces of the pressing element are adapted to the surface shapes of the respective end regions of the connecting elements.

Preferably the second pressing surface of the pressing element and/or the bottom surface of the second region of the U-shaped recess is/are provided with structures, which can penetrate into the surface of the elastic connecting element, and an optimal connection is thereby obtained.

Preferably at least the end region of the second connecting element is provided with structures that are designed in a way complementary to the structures of the second region of the U-shaped recess, whereby a formfitting connection is obtained.

An optimal formfitting connection of this kind is achieved if the structures consist of ribs and grooves that are aligned substantially transversely to the longitudinal axis of the second connecting element.

Preferably the clamping screw is connected to the pressing element in a rotatable way, which makes it possible to place the cap, the clamping screw screwed therein and the pressing element as a unit on the further bone screw, whereby the handling is considerably simplified.

Another advantageous embodiment of the invention consists in that at least on the side that co-operates with the second connecting element, the pressing element is provided with support surfaces that are supportable on stop faces provided on the arms. Thereby achieved is that the clamped end region of the elastic second connecting element is not subjected to too great a pressing.

An embodiment of the invention will be explained more closely in the following, by way of example, with reference to the attached drawings.

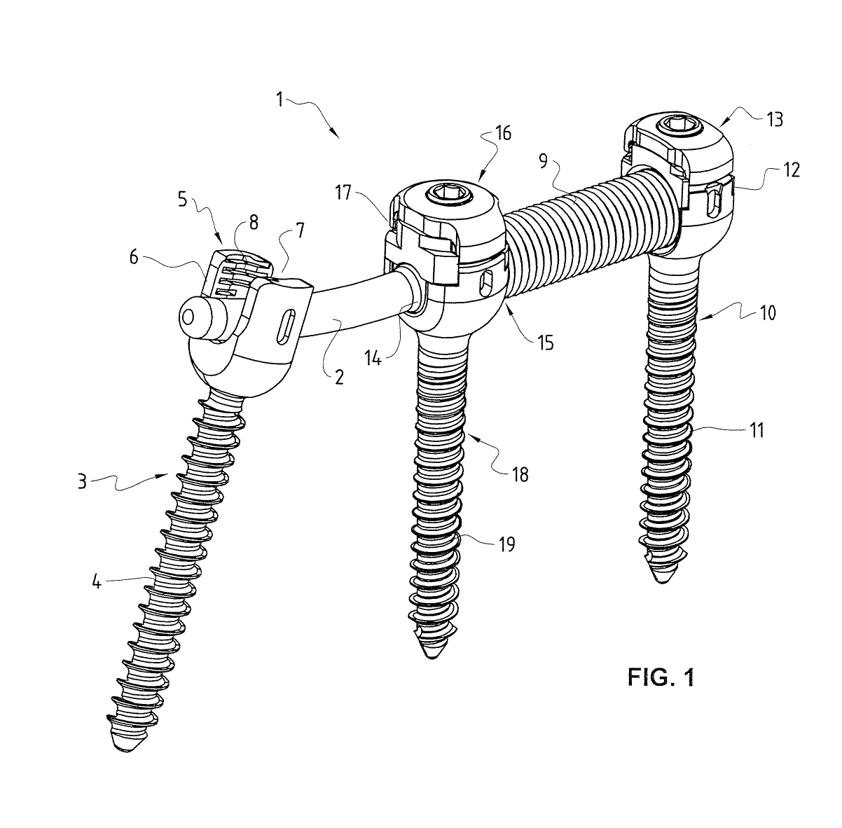

FIG. 1 shows in a three-dimensional representation a vertebral column implant, which comprises a first rigid connecting element, a second elastic connecting element and bone screws, in which the connecting elements are inserted and held, and a further bone screw, in which the end regions of the two connecting elements are inserted and fixed;

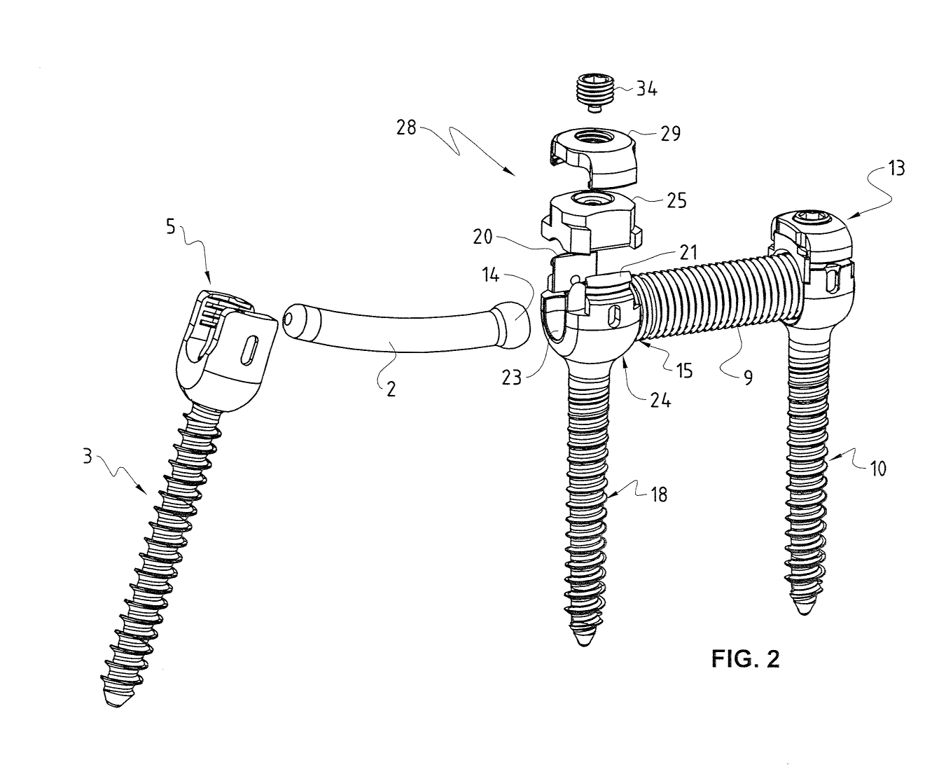

FIG. 2 shows in a three-dimensional representation the embodiment according to FIG. 1, the individual parts of the clamping means of the further bone screw being shown in taken-apart state;

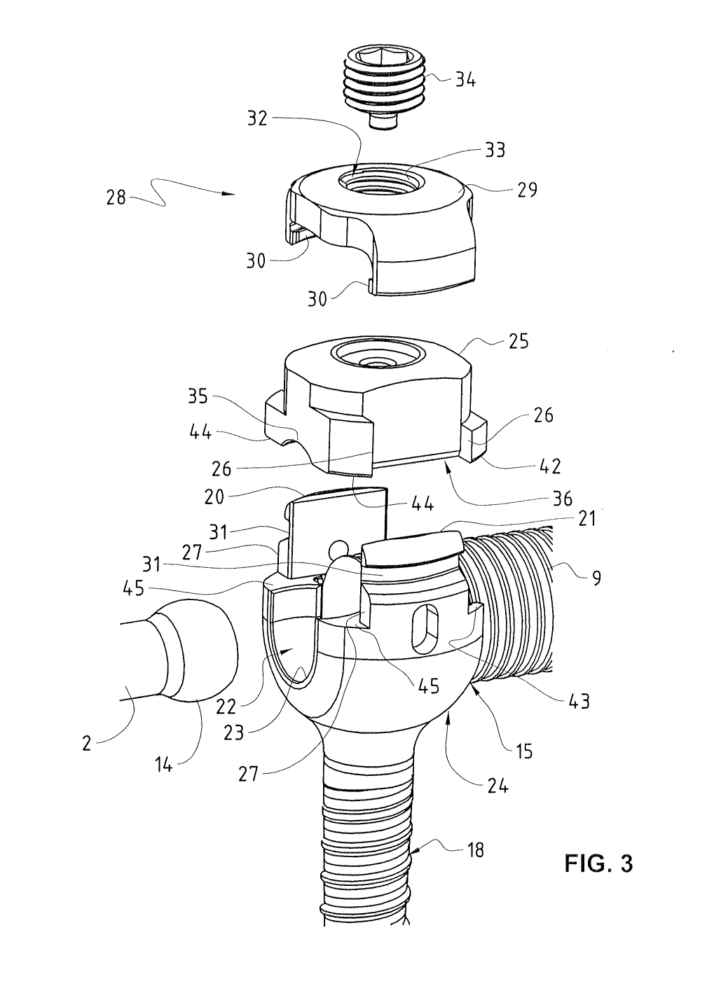

FIG. 3 shows in an enlarged illustration the receiving part for the connecting elements of the further bone screw according to FIG. 2;

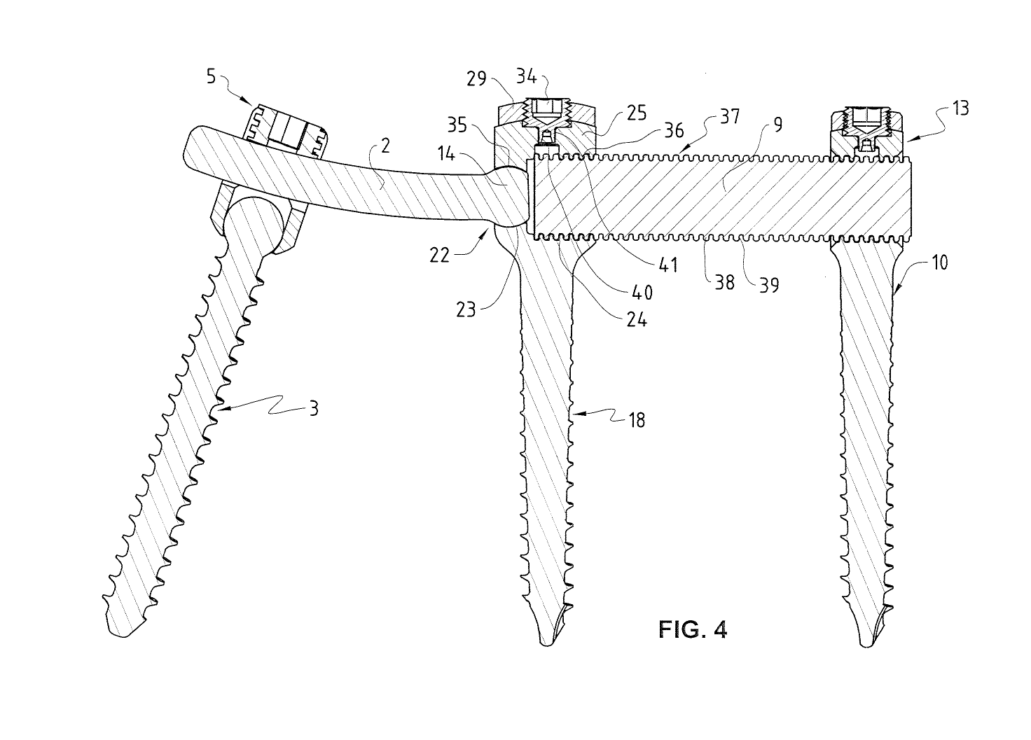

FIG. 4 shows a longitudinal sectional view of the vertebral column implant according to FIG. 1 and FIG. 2;

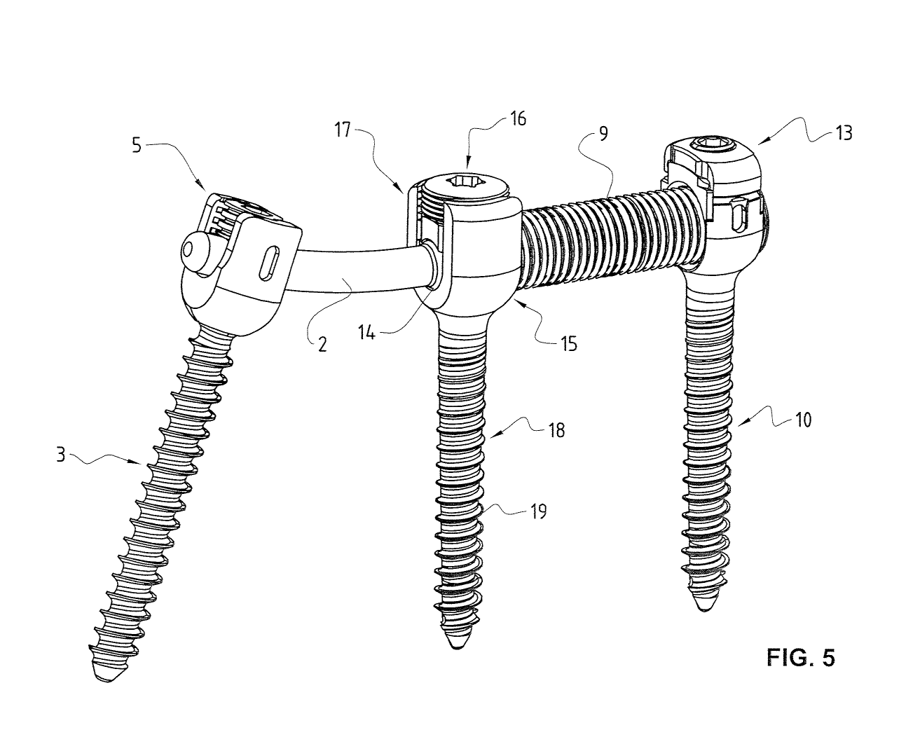

FIG. 5 shows in a three-dimensional representation another embodiment of a vertebral column implant according to the invention;

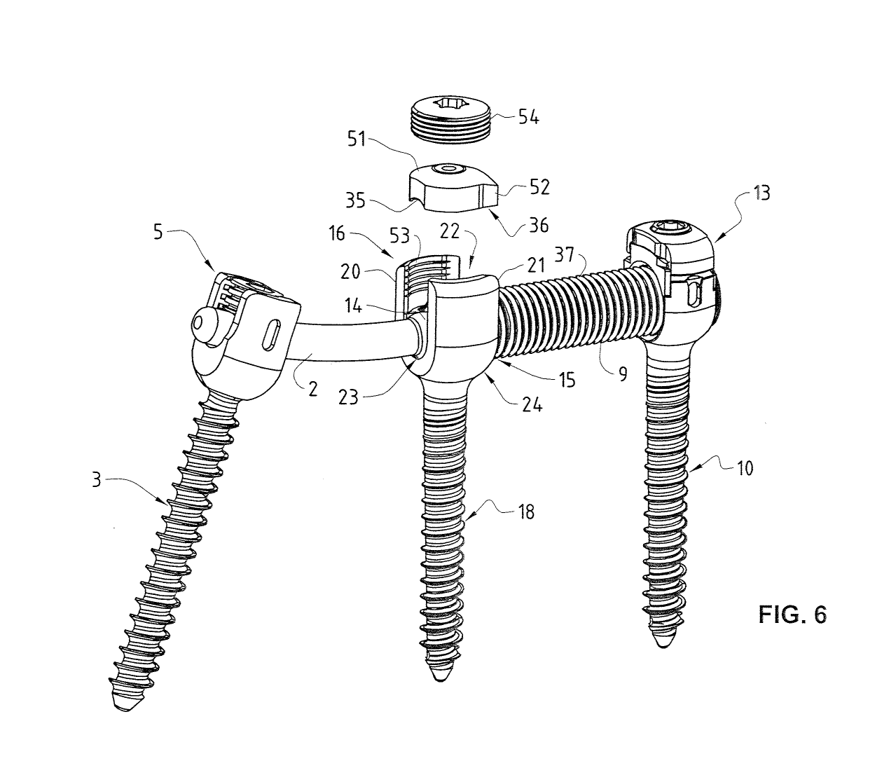

FIG. 6 shows in a three-dimensional representation the embodiment according to FIG. 5, the individual parts of the clamping means of the further bone screw being shown in taken-apart state; and

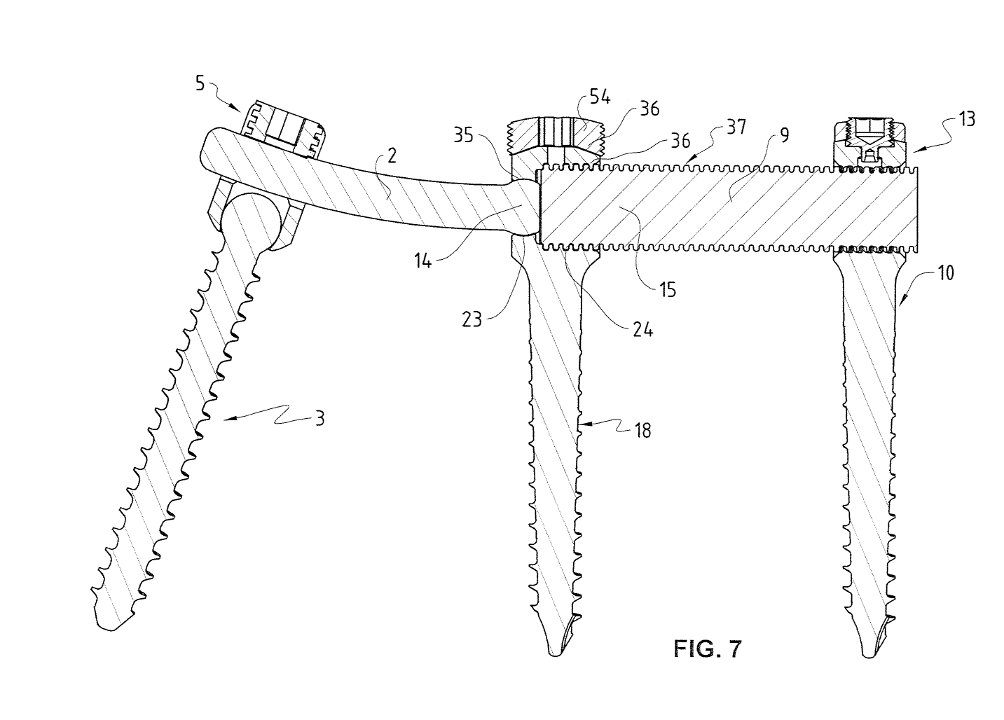

FIG. 7 shows a longitudinal sectional view of the vertebral column implant according to FIG. 5 and FIG. 6.

FIG. 1 shows in a three-dimensional representation a vertebral column implant 1 that has a first connecting element 2 which is rigid. This first connecting element 2 is held in a first bone screw 3, which comprises in a known way a screw-in part 4 that can be screwed into a vertebral body in a way likewise known (not shown). This first bone screw 3 is provided with first receiving means 5 for holding the first connecting element 2, which receiving means are designed as U-shaped receiving part 7 in the head part 6 of the first bone screw 3. The first connecting element 2, which is designed as a rod, can be placed in this U-shaped receiving part 7. The U-shaped receiving part 7 has on the inside a threading 8, into which a clamping screw can be screwed in a known way (not shown), by means of which screw the first connecting element 2 can be clamped in the U-shaped receiving part 7, which will be described in detail later.

The vertebral column implant 1 shown here further comprises a second connecting element 9, which is elastic and which is held in a second bone screw 10. This second bone screw 10 is likewise composed of a screw-in part 11 that can be screwed into a vertebral body in a known way (not shown). Disposed in the head part 12 of this second bone screw 10 are second receiving means 13, in which the second connecting element 9 can be fixed and held in the second bone screw 10. Such a second bone screw with the second receiving means for receiving the second connecting element is described in detail in the European patent application having the publication number 2 074 957, which is hereby referred to.

As can be seen from FIG. 1, the end region 14 of the first connecting element and the end region 15 of the second connecting element are held in a connecting way in connecting means 16. These connecting means 16 are disposed in the head part 17 of a further bone screw 18, which further bone screw 18 is likewise provided with a screw-in part 19 with which this further bone screw can also be screwed into a vertebral body in a known way (not shown).

As can be seen from FIG. 2 and in particular from FIG. 3, the connecting means 16 that are provided in the head part 17 of the further bone screw 18 comprise two arms 20 and 21 which form a U-shaped recess 22 in the head part 17. Insertable in this U-shaped recess 22 is in a first region 23 an end region 14 of the first connecting element 2. Inserted in a second region 24 of this U-shaped recess 22 is an end region 15 of the second connecting element.

Insertable between the two arms 20 and 21 is a pressing element 25. This pressing element 25 is provided with guides 26 in the form of projections. With placement of the pressing element 25 on the further bone screw 18, these guides 26 run along guide surfaces 27, which are provided on the flanks of each of the arms 20 and 21. The pressing element 25 can thereby be inserted in a guided way between the two arms 20 and 21 of the further bone screw 18. This pressing element 25 can be clamped by means of clamping means 28 against the end regions 14 and 15 of the first connecting element 2 or respectively of the second connecting element 9 when these end regions are inserted in the U-shaped recess 22. This clamping means 28 is made up of a cap 29 which can be placed on the two arms 20 and 21 and is lockable with these arms. For this purpose, the cap 29 has two ridges 30, which ridges 30 can be rotated in a known way in channels 31 provided on the outside of the arms 20 and 21. In the rotated-in state, the cap 29 is connected to the two arms 20 and 21.

The cap 29 is provided with a bore 32 which is provided with a threading 33. Screwable into the threading 33 of this bore 32 is a clamping screw 34. In the assembled state, as is shown in FIG. 1, the pressing element 25 can be clamped against the end regions 14 and 15 of the first connecting element 2 or respectively of the second connecting element 9 by tightening of this clamping screw 34, the pressing element 25 being pressed with a first pressing surface 35 against the end region 14 of the first connecting element 2 and with a second pressing surface 36 against the end region 15 of the second connecting element 9.

With the embodiment example shown in FIG. 1 to FIG. 4, the end region 14 of the first connecting element 2 is designed spherical. The bottom surface, formed between the first arm 20 and the second arm 21, of the first region 23 of the U-shaped recess 22 has a concave spherical shape that corresponds to the spherical end region 14 of the first connecting element 2, as can be seen in particular from FIG. 4. The first pressing surface 35 of the pressing element 25 likewise has a concave spherical surface that corresponds to the spherical shape of the end region 14 of the first connecting element 2. The spherical shape of the end region 14 of the first connecting element 2 can thereby be held and fixed in an optimal way in the further bone screw 18. It is thereby possible for the first connecting element 2 to be able to be angularly aligned with respect to the further bone screw 18 and thus to the second connecting element 9 within a certain range, an optimal clamping being nevertheless achieved.

As can also be seen from FIG. 4, the second connecting element 9 is provided with structures 37 on the surface. The second pressing surface 36 of the pressing element 25 and the bottom surface of the second region 24 of the U-shaped recess 22 are likewise provided with structures which are of complementary design to the structures 37 of the second connecting element 9. A formfitting connection to the second connecting element 9, which is composed of an elastic material, is thereby obtained in the further bone screw 18. Of course it would also be conceivable for the second connecting element 9 to have a smooth surface and only the bottom surface of the second region 24 of the U-shaped recess 22 and/or the second pressing surface 36 of the pressing element 25 to be provided with structures that would penetrate into the surface of the second connecting element 9 during tightening of the clamping screw 34 against the pressing element 25, whereby a formfitting connection would also be produced.

The structures 37 could be designed, for example, as ribs 38 and grooves 39, which could be aligned substantially transversely to the longitudinal axis of the second connecting element 9, whereby an optimal form fit would be created.

As can likewise be seen from FIG. 4, the clamping screw 34 can be held in a rotatable way in the pressing element 25, for which purpose this clamping screw 34 is provided with a bolt 40, which projects through a bore 41 made in the pressing element 25, the lower part of the bolt 40 being crimped, so that the clamping screw is held in a rotatable way in the pressing element 25. The clamping means 28, composed of cap 29 and clamping screw 34, can thereby be pre-assembled with the pressing element 25. These means can then be placed as a unit on the further bone screw 18, locked and correspondingly clamped, which considerably simplifies the manipulation during a surgical intervention.

As can be seen from FIG. 3, the pressing element 25 has support surfaces 42 on the side that co-operates with the second connecting element 9. Provided on the arms 20 and 21 are corresponding stop faces 43. The support surfaces 42 and the stop faces 43 are aligned in such a way with respect to one another that, with completely clamped state of the pressing element 25 and when a sufficient pressing force acts upon the second connecting element 9, which is made up of an elastic material, support surfaces 42 are supported on the stop faces 43, so that too great a pressing of the elastic second connecting element 9 in the clamping state is prevented.

The pressing element 25 can be provided with support surfaces 44 on the side that co-operates with the first connecting element 2, which support surfaces are supportable on stop faces 45 provided on the arms 20 and 21. The support surfaces 44 and the stop faces 45 can be harmonized with one another in such a way that, with inserted first connecting element 2, these surfaces and faces do not come into contact with one another. It is however conceivable that the first connecting element is removed when the stabilized vertebral bodies have developed a stable bone connection. When henceforth clamping the pressing element 25 on the second connecting element 9 only, the support surfaces 44 would then be supported on the stop faces 45. A stable clamping of the second connecting element 9 in the further bone screw 18 would nevertheless be able to be achieved.

Like the previously described example, the further embodiment of a vertebral column implant, as can be seen from FIG. 5 comprises a first rigid connecting element 2, which is held once again in a first bone screw 3. This first bone screw 3 likewise has first receiving means 5 for receiving the first connecting element 2, which is designed as a rod.

The elastic second connecting element 9 is held in a second bone screw 10, as has been described with reference to the previously presented embodiment example.

The end region 14 of the first connecting element 2 and the end region 15 of the second connecting element 9 are once again held in connecting means 16 in a connecting way. These connecting means 16 are disposed in the head part 17 of a further bone screw 18, which further bone screw 18 is likewise provided with a screw-in part 19 with which this further bone screw can likewise be screwed into a vertebral body in a known way (not shown).

As can be seen from FIG. 6 and FIG. 7, the connecting means 16, which are provided in the head part 17 of the further bone screw 18, again comprise two arms 20 and 21 which form a U-shaped recess 22 in the head part 17. Insertable in this U-shaped recess 22 in a first region 23 is an end region 14 of the first connecting element 2. Inserted in a second region 24 of this U-shaped recess 22 is an end region 15 of the second connecting element.

A pressing element 51 can be inserted between the two arms 20 and 21. This pressing element 51 is provided with a projection 52 which serves as guide during insertion of the pressing element 51 between the two arms 20 and 21. Provided on the inside on the two arms is a threading 53 into which a clamping screw 54 can be screwed. By tightening this clamping screw 54, the pressing element 51 can once again be clamped against the end regions 14 and 15 of the first connecting element 2 or respectively of the second connecting element 9. The pressing element 51 is thereby pressed once again with a first pressing surface 35 against the end region 14 of the first connecting element 2 and with a second pressing surface 36 against the end region 15 of the second connecting element 9.

Also with the further embodiment example presented here, the end region 14 of the first connecting element 2 is designed spherical. The bottom surface, between the first arm 20 and the second arm 21, of the first region 23 of the U-shaped recess 22 has a concave spherical shape that corresponds to the spherical end region 14 of the first connecting element 2, as can be seen in particular from FIG. 7. The first pressing surface 35 of the pressing element 25 likewise has a concave spherical surface that corresponds to the spherical shape of the end region 14 of the first connecting element 2. The spherical shape of the end region 14 of the first connecting element 2 can thereby be held and fixed in an optimal way in the further bone screw 18; it is thereby possible for the first connecting element 2 to be able to be angularly aligned within a certain range with respect to the further bone screw 18 and thus to the second connecting element 9, optimal clamping being nevertheless achieved.

In a way corresponding to that of the previously described embodiment example, structures 37 have also been provided on the second connecting element 9 in this further embodiment example too, in order to obtain a formfitting connection.

The elastic second connecting element used in vertebral column implants of this kind is preferably made of a polyurethane-based, biocompatible synthetic material, while the other parts are made of a titanium alloy. Of course other suitable materials can also be used.

Embodiments have been described in the preceding that have in each case been composed of a total of three screws, the first and the middle screw being connected to one another via a rigid connecting element, and the middle screw and the second screw being connected to one another via an elastic connecting element. Of course many other embodiment examples are conceivable having more screws, in which embodiments the rigid and elastic connecting elements extend over more than two screws, for example. It is also conceivable for more than two connecting element parts to be used; for instance, a rigid connecting element could be inserted between two elastic connecting elements, the previously described further bone screws being inserted in the region of the connections of these connecting elements. Thus a vertebral column implant put together in any desired way, depending upon need, can be used.

Obtained with this invention is a vertebral column implant for stabilization and stiffening of vertebral bodies of a vertebral column which can be adapted in an optimal way to the circumstances, which is easy to handle and with which a polyaxial adaptation of a rigid connecting element with respect to an elastic connecting element is likewise achievable in an optimal way.

1. A vertebral column implant for stabilization and stiffening of vertebral bodies of a vertebral column, comprising:
at least one first bone screw having a screw-in part that is screwable into a vertebral body, and first receiving means;
at least one second bone screw having a screw-in part that is screwable into a vertebral body, and second receiving means;
at least one first connecting element, which is rigid and is insertable into the first receiving means of the first bone screw and is able to be fixed therein;
at least one second connecting element, which is elastic and is insertable into the second receiving means of the second bone screw and is able to be fixed therein; and
connecting means, with which in each case a first connecting element is connectible to a second connecting element,
wherein the connecting means comprise two arms, which form a U-shaped recess and which are provided on a further bone screw, an end region of a first connecting element is insertable in a first region of the U-shaped recess, and an end region of a second connecting element is insertable in a second region of the U-shaped recess, a pressing element is insertable between the two arms, and clamping means are placeable on the arms, with which clamping means the pressing element is able to be pressed against said two end regions of the first and second connecting element.

2. The vertebral column implant according to claim 1, wherein the pressing element is provided with guides which co-operate with guide surfaces disposed on the flanks of the arms.

3. The vertebral column implant according to claim 1, wherein the clamping means comprise a clamping screw, with which the pressing element is able to be pressed with a first pressing surface against the end region of the first connecting element and a second pressing surface against the end region of the second connecting element.

4. The vertebral column implant according to claim 3, wherein the clamping screw is inserted into a cap, which is placeable on the arms and is lockable therewith.

5. The vertebral column implant according to claim 3, wherein the clamping screw is screwable into a threading provided on the inside of the arms.

6. The vertebral column implant according to claim 1, wherein the end region of the first connecting element is designed spherical and the bottom surface, formed between the first arm and the second arm, of the first region of the U-shaped recess has a concave spherical shape which corresponds with the spherical end region of the first connecting element.

7. The vertebral column implant according to claim 3, wherein the respective pressing surfaces of the pressing element are adapted to the surface shapes of the respective end regions of the connecting elements.

8. The vertebral column implant according to claim 3, wherein the second pressing surface of the pressing element and/or the bottom surface of the second region of the U-shaped recess is/are provided with structures.

9. The vertebral column implant according to claim 8, wherein at least the end region of the second connecting element is provided with structures that are designed in a way complementary to the structures of the second region of the U-shaped recess.

10. The vertebral column implant according to claim 8, wherein the structures consists of ribs and grooves that are aligned substantially transversely to the longitudinal axis of the second connecting element.

11. The vertebral column implant according to claim 3, wherein the clamping screw is connected to the pressing element in a rotatable way.

12. The vertebral column implant according to claim 1, wherein at least on the side that co-operates with the second connecting element, the pressing element is provided with support surfaces that are supportable on stop faces provided on the arms.
